(19) United States
(12) Patent Application Publication
(10) Pub. No.: US 2025/0034602 A1
(43) Pub. Date: Jan. 30, 2025

(54) COMPOSITIONS AND METHODS FOR MAKING MEDIUM CHAIN CARBOXYLIC ACIDS (MCCAs)

(71) Applicant: NUtech Ventures, Lincoln, NE (US)

(72) Inventors: Xu Li, Lincoln, NE (US); Qu Wen, Lincoln, NE (US); Qidong Yin, Lincoln, NE (US); Li Tan, Lincoln, NE (US)

(21) Appl. No.: 18/783,181

(22) Filed: Jul. 24, 2024

Related U.S. Application Data
(60) Provisional application No. 63/528,512, filed on Jul. 24, 2023.

(51) Int. Cl. C12P 7/40; C11C 1/08

(52) U.S. Cl. CPC C12P 7/40; C11C 1/08

(57) ABSTRACT

This document describes methods and compositions for converting organic wastes into medium chain carboxylic acids (MCCAs). This document also describes methods and compositions for obtaining high yields of caproate from a fermentation broth comprising MCCAs.

### CROSS REFERENCE TO RELATED APPLICATIONS

This application claims the benefit of priority under 35 U.S.C. § 119 (e) to U.S. Application No. 63/528,512 filed Jul. 24, 2023. This application is incorporated by referenced in its entirety.

### FEDERALLY SPONSORED RESEARCH OR DEVELOPMENT

This invention was made with government support under CBET1805990 awarded by the National Science Foundation (NSF) and under 2017-68003-26497 awarded by the U.S. Department of Agriculture, National Institute of Food and Agriculture. The government has certain rights in the invention.

### TECHNICAL FIELD

This disclosure generally relates to compositions and methods for making medium chain carboxylic acids (MCCAs).

### BACKGROUND

The use of waste as feedstock in biofuel and bioproduct manufacturing is considered one of the key pathways to achieve circular economy and sustainable development. Beef cattle manure represents a vast-yet-underutilized source of waste feedstock. In the US alone, about 3.7 billion kg of cattle manure were produced in 2020, accounting for 53.3% of all animal manure. Globally, only about 18.6% of animal manure were treated, while the remaining were left untreated or directly applied to cropland. Excess cattle manure in the field can lose nitrogen and other nutrients to runoffs and pollute surface water.

### SUMMARY

Producing and recovering bioproducts from organic wastes is an important part of building a circular economy. Livestock wastes can be a source of pollutants to surface and ground water, but they also represent a source of feedstock for production of bioproducts, such as medium chain carboxylic acids (MCCAs). This document describes the optimization of the conversion of organics in beef cattle manure to caproate and a novel extraction process using oleogel to selectively recover caproate from fermentation broth.

With intrinsic chain elongation bacteria in cattle manure, we achieved a maximum caproate production rate of 8.59 g/L/d and the maximum concentration of 11.09 g/L when ethanol was used as the electron donor. A diverse population of known chain clongators were detected in the reactor microbiome (e.g., Clostridium, Eubacterium, Caproiciproducens, etc.).

Oleogel beads were fabricated with aerogel and organic solvents (e.g., dodecane with 3% trioctylphosphine oxide). The direct contact between oleogel beads and fermentation broth enabled high mass transfer rates, achieving a cumulative caproate extraction efficiency of 87.39±0.96% in 180 min. In subsequent desorption process, caproate accounted for 86.18±0.32% of all carboxylic acids and alcohols desorbed from oleogel beads. Findings from this study demonstrate the technical feasibility of producing bioproducts from an abundant agricultural waste that would otherwise be a source of pollutant of the environment.

In one aspect, methods of making medium chain carboxylic acids (MCCAs; C6-C12) is provided. Generally, such methods include providing a feedstock, wherein the feedstock is cattle manure or wastewater from cattle manure holding ponds; combining the feedstock with material providing an electron donor at a suitable ratio; fermenting the combination of the feedstock with the material providing an electron donor under first appropriate conditions in fermentation broth to produce short chain fatty acids (SCFAs); elongating the SCFAs under second appropriate conditions into medium chain carboxylic acids (MCCAs); and extracting the MCCAs.

In some embodiments, the material providing the electron donor is corn silage. In some embodiments, the material providing the electron donor is ethanol, lactate, or combinations thereof. In some embodiments, the suitable ratio of the feedstock with the material providing an electron donor is from about 1:3 to about 1:5.

In some embodiments, the MCCAs comprise caproate. In some embodiments, the extraction of MCCAs is selective for caproate. In some embodiments, the extraction of caproate uses oleogel beads.

In some embodiments, the methods further include adding additional fermentation broth during or following the elongating step. In some embodiments, the methods further include adding additional material providing an electron donor to the additional fermentation broth.

In some embodiments, the method does not require exogenous enzymes for chain elongation. In some embodiments, the method relies on bacteria intrinsic to the feedstock for chain elongation. In some embodiments, the bacteria intrinsic to the feedstock are Clostridium, Eubacterium, Ruminococcaceae, or Caproiciproducens. In some embodiments, the method does not require pH adjustment.

In another aspect, systems for making medium chain carboxylic acids (MCCAs) are provided. Generally, such systems include a first reactor, wherein the input is cattle manure or wastewater from cattle manure holding ponds and the output is short chain fatty acids (SCFAs); and a second reactor, wherein the input is the SCFAs produced in the first reactor and the output is MCFAs. In some embodiments, the output from the second reactor is caproate.

In still another aspect, oleogel beads that include aerogel and at least one organic solvent are provided. In some embodiments, the organic solvent is immiscible with water and has low polarity. In some embodiments, the organic solvent is dodecane. In some embodiments, the organic solvent further comprises trioctylphosphine oxide (TOPO) at about 1.5% to about 6%. In some embodiments, the aerogel has a particle size of about 1.2 mm to about 4.0 mm. In some embodiments, the aerogel has a pore diameter of about 15 nm to about 25 nm.

In still another aspect, methods of obtaining high yields of caproate from a fermentation broth comprising MCCAs are provided. Such methods typically include contacting the fermentation broth comprising MCCAs with the oleogel beads described herein, thereby obtaining high recovery yields of caproate from the fermentation broth.

In one aspect, methods of making medium chain carboxylic acids (C6-C12; MCCAs) are provided. Such methods typically include providing a feedstock, wherein the feedstock is cattle manure or wastewater from cattle manure holding ponds; combining the feedstock with material providing an electron donor at a suitable ratio (e.g., 1:3 to 1:5); fermenting the combination under first appropriate conditions to produce SCFAs; fermenting the SCFAs under second appropriate elongation conditions to elongate the SCFAs into MCCAs; and extracting MCCAs, including caproate, from the fermentation broth.

In some embodiments, the intrinsic chain elongation bacteria are Clostridium, Eubacterium, or Caproiciproducens. In some embodiments, the methods described herein do not require exogenous enzymes for chain elongation. In some embodiments, the methods described herein do not require pH adjustment or require very little pH adjustment.

In some embodiments, the material providing the electron donor is selected from ethanol or lactate (from, e.g., corn silage).

In some embodiments, the methods described herein further include diluting the fermentation broth during the fermenting step. In some embodiments, the extraction of MCCAs is selective for caproate. In some embodiments, the extraction of caproate uses oleogel beads.

In another aspect, a system for making medium chain carboxylic acids (MCCAs) is provided. Such a system generally includes a first reactor, wherein the input is cattle manure and the output is short chain fatty acids (SCFAs); and a second reactor, wherein the input is SCFAs and the output is caproate.

In still another aspect, oleogel beads are provided that include aerogel and at least one organic solvent. In some embodiments, the organic solvent is dodecane. In some embodiments, the organic solvent is immiscible with water and has low polarity. In some embodiments, the organic solvent further comprises trioctylphosphine oxide (TOPO) at about 1.5% to about 6%.

In some embodiments, the aerogel has a particle size of about 1.2 mm to about 4.0 mm. In some embodiments, the aerogel has a pore diameter of about 20 nm.

In yet another aspect, methods of obtaining high yields of caproate from a fermentation broth comprising MCCAs are provided. Such methods typically include contacting the fermentation broth comprising MCCAs with the oleogel beads described herein, thereby obtaining high recovery yields of caproate from the fermentation broth.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1C are experimental data showing SCCAs and MCCAs production using cattle manure during the fermentation and chain elongation stages. (1A) Concentrations of SCCAs, MCCAs, and ethanol during Stages I and II. (1B) Caproate production during Stage II simulated using the Modified Gompertz model. The dots represent the mean values from triplicate reactors at each time point and the dotted lines represent the simulation results. (1C) The product selectivity, specificity, and yield of caproate at the end of Stage II.

FIGS. 2A-2B are experimental data showing the effect of dilution and second ED dosage on chain elongation during Stage III and IV. (2A) Concentrations of SCCAs, MCCAs, and ethanol during stages III and IV. (2B) Concentrations of LCAs during stages III and IV.

FIGS. 3A-3C are experimental data showing characterization of the microbial communities during the four stages of reactor operation. (3A) Beta diversity based on weighted UniFrac distance, where the color and the size of the dots distinguish groups and times, respectively; (3B) Microbial community structure at the phylum level (top 15 phyla); (3C) Annotated ASVs containing chain elongators at the genus level.

FIGS. 4A-4C are experimental data showing the performance of oleogel beads on the extraction of caproate from real manure chain-elongation broth and subsequent desorption to pH 12 solutions. (4A) Image of pristine aerogel and fabricated oleogel. (4B) Concentrations of ethanol, SCCAs, and MCCAs in the chain elongation broth during the extraction with oleogel beads. (4C) Concentrations of ethanol, SCCAs, and MCCAs in the pH 12 solutions during the desorption with oleogel beads.

FIGS. 5A-5B are experimental data showing complex organics during the fermentation stage (stage I). (5A) Concentration of soluble carbohydrate on day 0 and day 7. (5B) Concentration of protein on day 0 and day 7.

FIG. 6 is graphical data showing changes in pH at the beginning and end of 4 stages.

FIG. 7 is experimental data showing changes in LCAs during fermentation and chain elongation stages.

FIGS. 8A-8C show predicted functions based on the PICRUSt2. (8A) Heatmap of identified genes involved in the RBO pathway and genes involved in acetyl-CoA or LCA production. The blue-to-red colors represent the relative abundance of genes. The abbreviation before the line in the column name represents the group (Con, E1, E3, and E5), and the number after the line represents the sampling days. The full gene name and description of each KO can be found in Table 3. (8B) Total relative abundance of genes involved in the RBO pathway. (8C) Total relative abundance of genes involved in acetyl-CoA or LCA production.

FIGS. 9A-9B show predicted fatty acid biosynthesis pathway based on the PICRUSt2. (9A) Heatmap of identified genes involved in the fatty acid biosynthesis pathway. The blue-to-red colors represent the relative abundance of genes. The abbreviation before the line in the column name represents the group (Con, E1, E3, and E5), and the number after the line represents the sampling point (day). (9B) Total relative abundance of genes involved in the fatty acid biosynthesis pathway.

FIG. 10 shows percentages of caproate before the extraction (chain elongation broth) and after the desorption (1st, 2nd, and 3rd desorption) from oleogel beads.

### DETAILED DESCRIPTION

The conversion of organic wastes into medium chain carboxylic acids (MCCAs; also referred to herein as medium chain fatty acids (MCFAs)) has recently received much attention due to the high economic values and wide range of applications of MCCAs, compared to common fermentation products, e.g., biogas and short chain carboxylic acids (SCCAs; also referred to herein as short chain fatty acids (SCFAs)). MCCAs are defined as carboxylic acids with an aliphatic straight carbon chain of 6 to 12 carbon atoms and can be produced through chain elongation by chain elongating bacteria such as Clostridium, Meghsphaera, and Eubacterium via the reverse β-oxidation pathway. When electron donors (ED) (e.g., ethanol or lactate) are available, chain elongators add two carbon atoms to the carboxylic acid chain (electron acceptors, or EA) during each reverse β-oxidation cycle, converting SCCAs into MCCAs (e.g., acetate to butyrate and then to caproate). MCCAs can be used to produce food additives, fragrances, pharmaceuticals, antimicrobials, lubricants, surfactants, and even biofuel. Caproate, one of the most attractive MCCAs, is estimated to have a refined value of $2,000-$3,000 per ton.

Biologically produced MCCAs from organic wastes often exist in a liquid mixture with other organic compounds such as SCCAs. Although the hydrophobicity of MCCAs leads to easier extraction from fermentation broth than SCCAs, the downstream separation is still a limiting factor and may account for 40 to 50% of the total cost of the waste-to-value production processes. To recover MCCAs from the chain elongation broth, simple and economical separation processes are needed. Some technologies have been tested for MCCA extraction, such as electrodialysis, ion exchange, and membrane system (e.g., supported liquid membrane and pertraction). However, these technologies face varying degrees of poor selectivity, low efficiency, and high cost. For example, electrodialysis cannot selectively extract MCCAs from chain-elongation broth without significant co-extraction of SCCAs; ion exchange requires demanding conditions for the subsequent stripping process; and, for membrane systems, membrane fouling can be a significant issue. Therefore, developing an efficient and economic technology to recover MCCAs from chain-elongation broth is needed to implement the waste-to-value production processes in real-world applications.

The objectives of this work were to (1) optimize the conversion of organics in beef cattle manure to caproate and (2) develop a novel extraction process to selectively recover caproate from chain-elongation broth. Beef cattle manure can be used to produce high titer and yield of MCCAs in single reactor systems. The microbiome and functional populations inside the bioreactors were characterized using high-throughput sequencing. Furthermore, we fabricated oleogel beads to develop a simple and fast process to extract caproate from real manure chain elongation broth with high efficiency and selectivity. The findings from this study establish a comprehensive process to tap the vast potential of an abundant source of organic wastes for MCCA production.

As described herein, cattle manure or wastewater from cattle manure holding ponds are superior feedstocks for caproate production. We demonstrated that, compared to other waste organics, cattle manure is a superior feedstock for caproate production, due not only to the high organic content but also to the intrinsic microbes that possess a high efficiency for chain elongation during fermentation. For example, using activated sludge as feedstock, microbes intrinsic to the activated sludge achieved a maximum caproate production rate of 0.64-0.97 g COD/L/d and a maximum concentration of 5.21-8.22 g/L. In a study using corn beer and corn silage as the feedstocks and microbes from an anaerobic digester treating corn silage as inoculum, the caproate production rate ranged from 0.1-1.7 g COD/L/d. In contrast, the methods described herein achieved a maximum caproate production rate of 18.98 g COD/L/d and a maximum titer of 25.66 g COD/L without much lag time. A mixed culture study achieved 10.0 g COD/L/d caproate production rate using butyrate as the EA and lactate as the ED after adopting the “Design-Build-Test-Learn” approach, while caproate production rate was 90% faster with microbes intrinsic to cattle manure (18.98 vs 10.0 g COD/L/d, or 8.59 vs 4.55 g/L/d). Furthermore, the caproate yield achieved with an ED:EA ratio of 3:1 using the methods described herein was also higher (0.40 vs 0.13-0.32 g caproate/g COD) than that using other organic wastes (e.g., acidified swine manure, Chinese liquor-making wastewater, and municipal solid waste) and mixed microbial cultures. Besides, other waste-fed MCCA-production systems usually formed substantial amounts of heptanoate and caprylate along with caproate. In contrast, cattle manure produced caproate as the most dominant MCCA (98.22-99.95% at the end of Stage II), making it easier downstream purification process.

The robust caproate production using cattle manure is partially attributed to the microbiome that is intrinsically efficient in fermentation and chain elongation. Many caproate producers were isolated from rumen of cattle and sheep. For instance, Clostridium kluyveri was isolated from the bovine rumen and could produce substantial levels (12.8 g/L) of caproate using ethanol and acetate in vitro. A previous study found similar abundance of Clostridium spp. in rumen and feces of cattle. Other caproate producers including Eubacterium limosum, Eubacterium pyruvativorans, Megasphaera elsdenii, and Megasphaera hexanoica were also initially isolated from sheep or cow rumen samples. The wide occurrence of caproate producers in rumen lead to high abundance of caproate producers in manure, which is important to the valorization of cattle wastes.

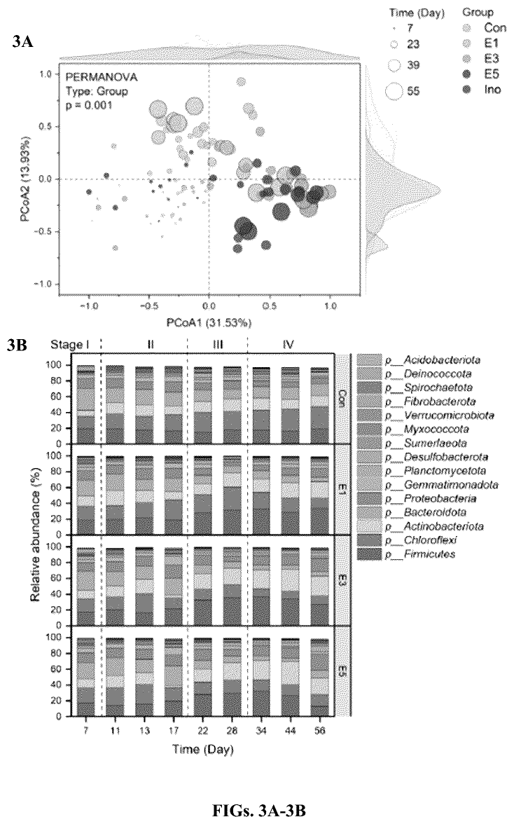

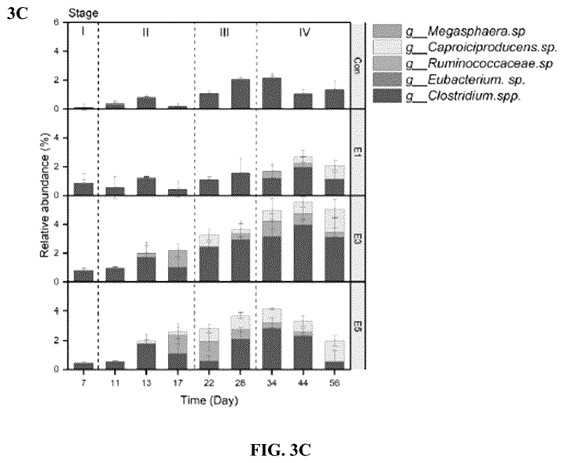

In addition, product inhibition shifted the chain elongation pathway. Following dilution, LCA production started when extra ED was provided (FIG. 3B). LCAs could be produced via either acetone-butanol-ethanol (ABE) fermentation, Wood-Ljundahl pathway with syngas, or corresponding carboxylate reduction. Since carbohydrate was not the main component of cattle manure and no ethanol was simultaneously formed, LCAs were less likely to be produced via ABE fermentation. Besides, LCAs could hardly be formed via the Wood-Ljundahl pathway because no extra CO₂ was supplemented. Therefore, LCA production was more likely to be generated via corresponding carboxylate reduction with the consumption of syngas (e.g., H₂, CO₂, and CO).

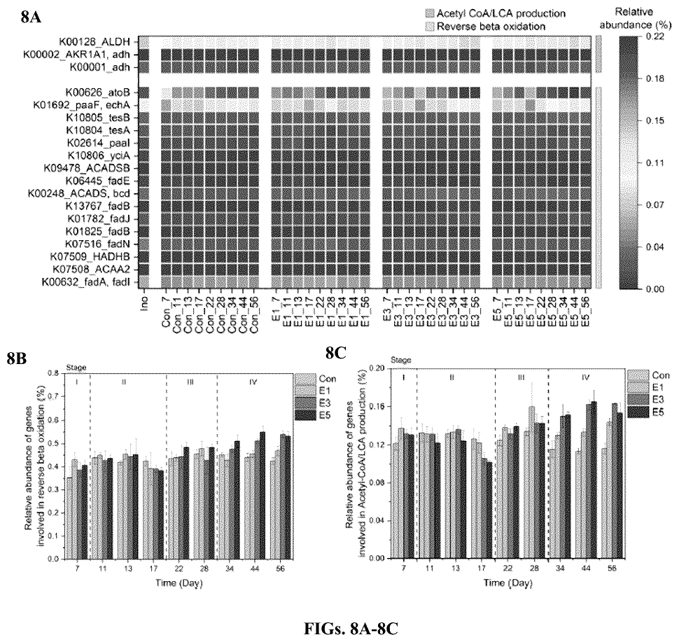

At Stage IV, LCAs production (butanol and hexanol) was accompanied by excess ethanol oxidation and carboxylate reduction (butyrate and caproate). This phenomenon also supported the hypothesis that LCA was formed via corresponding carboxylate reduction. With ED:EA ratios of 3:1 and 5:1, H₂ could be first formed when ethanol was converted into acetate and then H₂ was utilized to reduce carboxylate into alcohols. Consistently, the relative abundance of genes involved in the LCAs production (e.g., aldehyde dehydrogenase and alcohol dehydrogenase) increased in ED:EA ratios of 3:1 and 5:1 at Stage IV (FIG. 8). These results suggested that the microbial activity of caproate production could be hardly recovered once the product inhibition occurred. Instead, the microbiome tended to shift the MCCA production pathway into either the first chain elongation to butyrate or the corresponding LCA production.

Based on the experiments described herein, highly efficient methods of making medium chain carboxylic acids (MCCAs) and, in particular, caproate, are described. Briefly, the methods include providing a feedstock such as cattle manure or wastewater from cattle manure holding ponds and combining the feedstock with material that provides an electron donor at a suitable ratio. Electron donors can come from any number of materials, including, without limitation, corn silage or components thereof (e.g., ethanol, lactate, or combinations thereof). In addition, as demonstrated herein, the suitable ratio of the feedstock with the material providing an electron donor is from about 1:3 to about 1:5 (e.g., 1:3; 1:4; 1:5).

The methods described herein also include fermenting the combination of the feedstock with the material providing an electron donor under first appropriate conditions in fermentation broth to produce short chain fatty acids (SCFAs) and elongating the SCFAs under second appropriate conditions into medium chain carboxylic acids (MCCAs). First appropriate conditions for fermentation are described in detail in Example 1 and generally include, without limitation, growth under anaerobic conditions and optionally can include the addition of a methanogenesis inhibitor (e.g., 2-bromoethanesulfonic acid (BES)). Second appropriate conditions for elongation are described in detail in Example 1 and generally include determining the appropriate amount of electron donor material, which typically is dependent upon the amount of SCFAs present.

In some instances, it may be desirable to add additional media (i.e., dilute) and/or additional material providing an electron donor to the fermentation broth. The addition of either or both can result in the continued production of SCFA and, hence, the continued production of MCCAs, which ultimately can optimize the production of caproate.

As described herein, the methods can rely on the presence of bacteria that is intrinsic to the feedstock (e.g., endogenous bacteria) for chain elongation, which can result in significant cost savings when compared to methods that require the addition of exogenous elongating enzymes or exogenous bacteria that express, naturally or recombinantly, the elongating enzymes. In some embodiments, the bacteria that is intrinsic to the feedstock is bacteria from the genus Clostridium, Eubacterium, Ruminococcaceae, or Caproiciproducens. Depending on the feedstock used, the methods described herein may or may not require a pH adjustment.

The methods described herein include extracting the MCCAs. It would be appreciated that caproate is an MCCA, and, as described in more detail below, the extraction method can be selective for caproate. As described in more detail below, caproate can be extracted using olcogel beads.

The methods described herein can be performed using a system. For example, a system can include a first reactor and a second reactor. The input into the first reactor can be, without limitation, cattle manure or wastewater from cattle manure holding ponds, and the output from the first reactor can be, without limitation, short chain fatty acids (SCFAs). Similarly, the input into the second reactor can be, without limitation, SCFAs, and the output from the second reactor can be, without limitation, MCCAs. Specifically, the output from the second reactor can be, without limitation, caproate.

In addition, and also as described herein, oleogel extraction was developed as a novel technology for caproate recovery. The recovery of MCCAs from chain elongation broth is critical to the implementation of the waste-to-value production processes in real-world applications. In this study, the fabrication process of oleogel beads was simple and fast and was completed at ambient temperature and pressure. Due to the porous ultralight gel structure of aerogel (containing 99.8% air), the fabricated oleogel beads had a porous solid network dominated with solvent pockets.

The high absorption rate of oleogel beads could be attributed to (1) the small pore sizes on oleogel surface (i.e., 20 nm in aerogel vs. 45 μm in the supported liquid membrane) and (2) the high surface-to-volume ratio (600-800 m²/g) of aerogel beads with a diameter of 1.2-4.0 mm. Also, the extraction of caproate using oleogel didn't require transmembrane transportation, which is a rate-limiting step when using membrane materials to extract MCCAs.

Unlike membrane-based MCCA extraction methods, oleogel beads do not have the problem of membrane fouling. To alleviate membrane fouling in membrane systems, a pre-filter device or gas bubbling is often required. However, these measures increase either the operating costs or the space occupation of treatment units. In addition, pertraction systems require pumping systems to circulate a large volume of the viscous organic liquid between forward and backward membranes to keep the mass transfer rate sufficiently high. Oleogel beads provide a potentially economical option without encountering the above problems.

As described herein, oleogel beads that include aerogel and at least one organic solvent can be used to extract the caproate from the MCCAs. In general, the at least one organic solvent is immiscible with water and has low polarity. A representative organic solvent that can be used in the extraction of caproate is dodecane. In some instances, the yield of caproate can be increased by adding about 1.5% to about 6% (e.g., about 1.75% to about 5.5%, about 2% to about 5%, about 3% to about 4%, about 3.5%) trioctylphosphine oxide (TOPO) to the organic solvent. As described herein, the aerogel can have a particle size of about 1.2 mm to about 4.0 mm (e.g., about 1.5 mm to about 3.5 mm, about 2 mm to about 3 mm, about 2.5 mm) and a pore diameter of about 15 nm to about 25 nm (e.g., about 18 nm to about 22 nm; about 19 nm to about 21 nm; about 20 nm).

The oleogel beads described herein can be used to obtain high yields of caproate. Typically, the oleogel beads are used to obtain high yields of caproate from a fermentation broth that includes MCCAs.

In accordance with the present invention, there may be employed molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### Examples

### Example 1—Materials and Methods

Beef Cattle Manure Collection. Beef cattle manure collected from the Eastern Nebraska Research, Extension, and Education Center in Mead, NE, USA was used as feedstock for the bioreactors. Fresh manure was sieved through a 1 cm×1 cm mesh sieve, air dried, and stored at 4° C. until use. The total solids (TS) and volatile solids (VS) of cattle manure were 67.34% and 13.47%. The other components identified in cattle manure are shown in Table 1.

TABLE 1
Summary of cattle manure features

| Feature | Dry basis |
|---|---|
| Organic N, % N | 0.55 |
| Ammonium, % N | 0.008 |
| Nitrate, % N | <0.001 |
| Total N (TKN), % N | 0.56 |
| Phosphorus, % P2O5 | 0.74 |
| Potassium, % K2O | 0.90 |
| Sulfur, % S | 0.14 |
| Calcium, % Ca | 1.22 |
| Magnesium, % Mg | 0.56 |
| Sodium, % Na | 0.14 |
| Zinc, ppm Zn | 101.2 |
| Iron, ppm Fe | 13457.2 |
| Manganese, ppm Mn | 518.9 |
| Copper, ppm Cu | 22.6 |
| Soluble salts, mmho/cm | 12.48 |
| pH | 7.9 |
| Dry matters (TS), % | 67.3 |
| VS, % | 20.0 |

Chain Elongation Experiment. The chain elongation experiments were conducted in twelve 120 mL serum bottles and consisted of 4 stages: (I) fermentation, (II) chain elongation, (III) dilution, and (IV) second ED dosage. At Stage I (fermentation stage, Day 0-7), each bottle was filled with 41.70 g wet weight of cattle manure and 83.30 mL of water to achieve an initial solid content of 4.49% VS (w/w %). 10.00 g/L 2-bromoethanosulfonic acid (BES) was added as a methanogenesis inhibitor. The original pH was adjusted to 7 with 1M HCl. Each serum bottle was flushed with nitrogen gas for 3 min to ensure the anaerobic environment and then was sealed with a rubber stopper. Then, the serum bottles were placed on a shaker at 200 rpm under 30±1° C. Liquid and gas samples were periodically collected for analyses.

At Stage II (chain elongation stage, Day 8-17), 12 bottles were divided into 4 groups with 3 bottles in each group as triplicates. Based on the concentration of EA (SCCAs: acetate, propionate, iso-butyrate, butyrate, iso-valerate, valerate) at the end of Stage I, various amounts of ethanol were supplemented to achieve ED:EA ratios of 1:1, 3:1, and 5:1 (mol C) in E1 (3.84±0.40 g/L), E3 (11.73±0.62 g/L), and E5 (20.29±0.95 g/L) groups, respectively. The fourth group without ethanol addition was designated as the control group (Con).

At the beginning of Stage III (dilution stage, Day 18-28), half of the well-mixed manure slurry was replaced with the same volume of tap water on Day 18. BES was supplemented to maintain at the same level as in previous stages. After the 2-fold dilution, each serum bottle was flushed with nitrogen gas for 3 min to ensure an anaerobic environment.

At Stage IV (second ED dosage, Day 29-56), based on the concentrations of EA at the end of Stage III, various amount of ethanol was re-supplemented in E1, E3, and E5 groups to achieve initial ED:EA ratios of 1:1, 3:1, and 5:1 (mol C), respectively.

High-Throughput Sequencing. Metagenomics based on the 16S rRNA gene was conducted to determine the microbial composition in the reactors. Detailed procedure for sequencing can be found in the Supplementary Information file.

Oleogel Fabrication. Aerogel beads (Lumira Translucent Aerogel, LA1000) were purchased from CABOT (Massachusetts, USA). The particle size of aerogel beads ranged from 1.2-4.0 mm, with a pore diameter of about 20 nm. Other features of aerogel beads reported by the manufacturer are shown in Table 2. The organic solvents to be embedded in the aerogel beads were prepared by dissolving 3% TOPO into dodecane (Sigma-Aldrich, Massachusetts, USA) via vigorous mixing. Dodecane and TOPO have been used as extractants for hydrophobic organic molecule extraction in membrane-based system. Oleogel beads were fabricated by immersing the aerogel beads into the prepared solvents at room temperature for 2 h. After the pores of the aerogel were filled with solvents, the aerogel beads became oleogel beads and turned transparent. Then oleogel beads were taken out and the residual solvents on the oleogel surface were carefully removed by cool compressed air.

TABLE 2
Summary of aerogel bead features

| Property | LA1000 | |
|---|---|---|
| Particle size range | 1.2-4.0 | mm |
| Pore diameter | About 20 nm | |
| Porosity | >90% | |
| Particle density | 120-150 | kg/m3 |
| Surface chemistry | Hydrophobic | |
| Surface area | 600-800 | m2/g |

| | |
|---|---|
| CAS Registry Number | 102262-30-6 |

Oleogel Extraction Experiment. Real chain elongation broth was used to test the extraction of oleogel beads for caproate. Before the extraction, the pH of the liquid was adjusted to 4, making caproic acid the dominant species. Then, 0.8 g oleogel beads were added to 2 mL of pH-adjusted broth at room temperature for 180 min.

To characterize the desorption of MCCAs from oleogel beads, oleogel beads were rinsed in 2 mL of DI water for 1 min before being transferred to 2 mL sodium hydroxide solution at pH 12. The difference in caproate concentration in the oleogel beads and alkaline solutions resulted in a concentration gradient, driving the caproate desorption from oleogel beads. After 90 min, the oleogel beads were transferred to fresh 2 mL of alkaline solutions for another 90 min of desorption. The oleogel beads were transferred one more time to fresh 2 mL of alkaline solutions for a last 60 min. The total desorption time was 240 min. All experiments were carried out in triplicates.

Chemical Analysis. Alcohols, SCCAs, and MCCAs were measured using a gas chromatograph (GC7820A, Agilent, California, USA) coupled with a flame ionization detector and a capillary column. Detailed analytical procedure can be found in the Supplementary Information file.

Calculation. The modified Gompertz model was applied to simulate the dynamic parameters of MCCAs production. Product yield, selectivity, specificity, and oleogel extraction efficiency were calculated. Detailed calculations could be found in the Supporting Information.

Sequences. The sequences generated were deposited in the NCBI Sequence Read Archive (SRA) with the accession number PRJNA913948.

### Example 2—Results

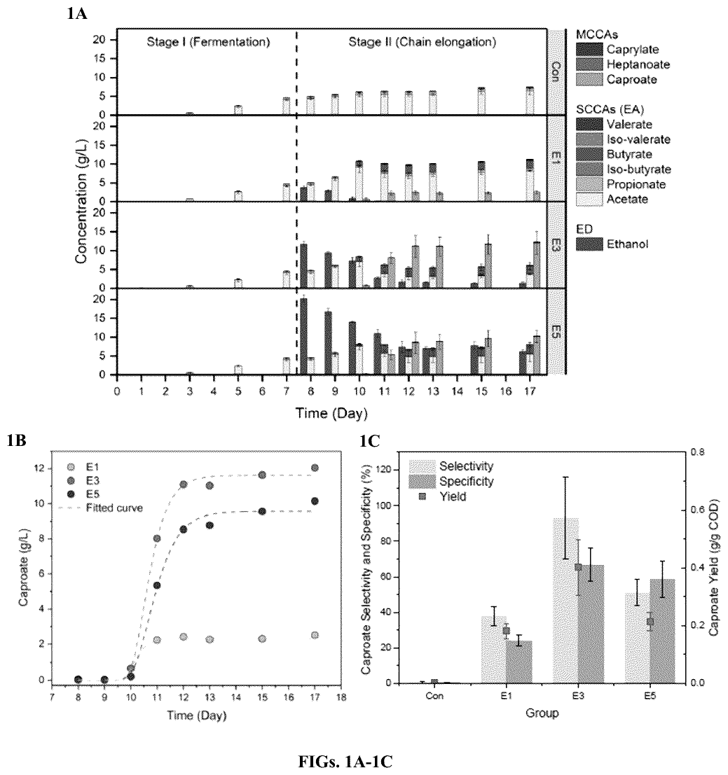

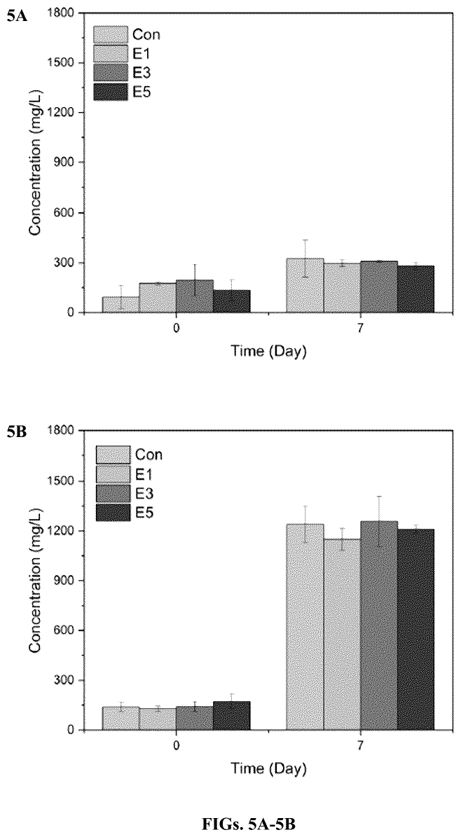

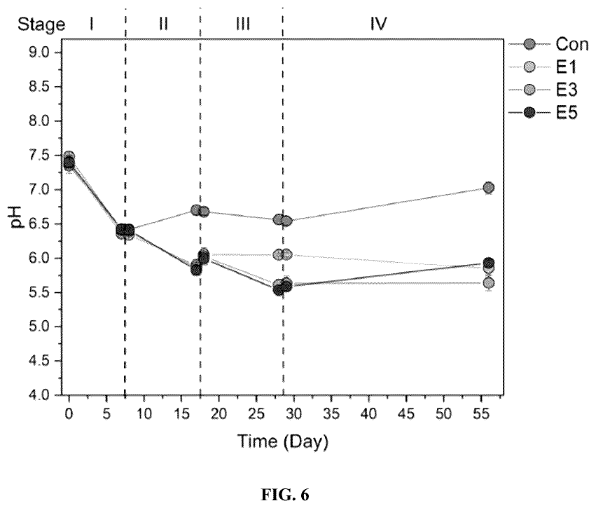

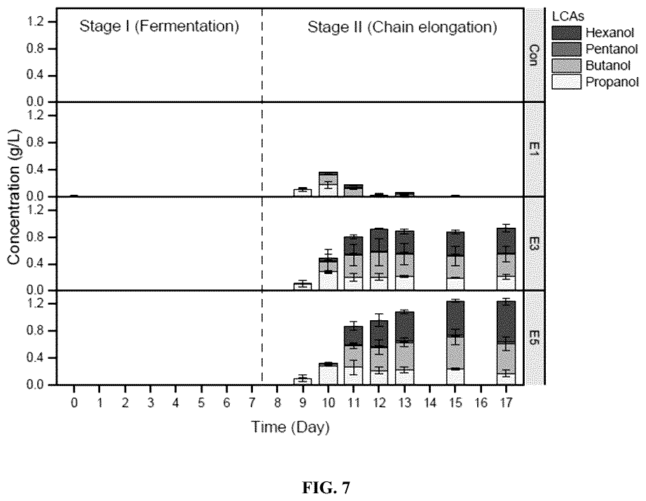

MCCA Production from Cattle Manure (Stage I & II). Batch reactors were operated in stages to produce SCCAs via fermentation and to produce MCCAs via chain elongation. In Stage 1 (fermentation, Day 0-Day 7), complex organics in cattle manure were hydrolyzed to small organic compounds such as SCCAs acetate and butyrate. Soluble carbohydrate and protein increased from 0.15±0.07 g/L and 0.15±0.03 g/L on Day 0 to 0.30±0.05 g/L and 1.21±0.10 g/L on Day 7, respectively (FIG. 5). No CH₄ and only trace amount of H₂ (<0.6 mL) were produced. Acetate was the most dominant product (4.05±0.24 g/L, equivalent to 4.33±0.26 g COD/L), followed by small amounts of propionate and butyrate (FIG. 1A). Due to the accumulation of SCCAs, pH decreased from 7.41±0.09 on Day 0 to 6.41±0.05 at the end of Stage I (FIG. 6). All SCCAs were considered as EAs to calculate the amount of ethanol needed as ED in Stage II.

At stage II (chain elongation, Day 8-Day 17), different amounts of ethanol were added to reactors to achieve three initial ED:EA carbon molar ratios of 1:1 (E1 group), 3:1 (E3 group), and 5:1 (E5 group). Chain elongation started right after ethanol addition. Caproate was detected on Day 10 and reached high levels for all three groups on Day 12 (FIG. 1A). A maximum caproate concentration of 2.41±0.47 g/L (5.33±1.04 g COD/L), 11.09±2.96 g/L (24.51±5.33 g COD/L), and 8.53±2.90 g/L (18.84±5.24 g COD/L) was achieved in the E1, E3, and E5 groups, respectively (FIG. 1A). The Modified-Gompertz model satisfactorily described the dynamics of caproate production (FIG. 1B, R²=0.99). The simulated maximum caproate concentration (Cₘₐₓ) for E1, E3, and E5 was 2.37 g/L (5.23 g COD/L), 11.61 g/L (25.66 g COD/L), and 9.58 g/L (21.17 g COD/L), respectively. Caproate measured in E3 was close to the solubility of caproate in water (10.80 g/L). Besides, the maximum caproate production rate (Rₘₐₓ) for group E3, 8.59 g/L/d (18.98 g COD/L/d), was higher than the other two groups tested, 2.69 g/L/d (5.94 g COD/L/d) for E1 and 5.82 g/L/d (12.86 g COD/L/d) for E5.

After reaching the plateau levels, the formation of caproate slowed down. For E1, this was mainly due to the depletion of ED (ethanol). For E3 and E5, both ED (ethanol) and EA (e.g., acetate and butyrate) still existed, suggesting that the toxic effects of undissociated caproate (i.e., caproic acid) under low pH might have inhibited further production of caproate. The pH of E3 and E5 dropped from 6.39±0.01 and 6.41±0.02 on Day 8 to 5.87±0.12 and 5.83±0.06 on Day 17. Under such low pH conditions, the final concentration of undissociated caproate in E3 and E5 were calculated to be 1.03±0.06 g/L and 0.96±0.04 g/L, well exceeding its toxicity threshold (about 0.2 g/L) reported for chain elongators.

At the end of Stage II, different yield, selectivity, and specificity of caproate occurred among the E1, E3, and E5 groups (FIG. 1C). The ultimate yield of caproate in E1, E3, and E5 groups (Equation 2) was 0.18±0.03 g/g COD (0.40±0.06 g COD/g COD), 0.40±0.10 g/g COD (0.89±0.21 g COD/g COD), and 0.21±0.03 g/g COD (0.47±0.07 g/g COD), respectively. The selectivity of caproate production (Equation 3) in E1, E3, and E5 groups was 37.95±5.55%, 93.03±22.85%, and 51.09±7.40% (in mol C), respectively. The specificity of caproate production (Equation 4) in E1, E3, and E5 groups was 23.95±3.15%, 66.66±9.38%, and 58.58±10.32%, respectively. E3 exhibited higher yield, selectivity, and specificity than the other two conditions. The caproate yield based on ethanol in E1, E3, and E5 groups was 0.27±0.03, 0.47±0.12, and 0.29±0.05 mol (caproate)/mol (ethanol), respectively. Supplementing ethanol with ED:EA ratio of 3:1 nearly achieved the theoretical caproate yield of 0.5 mol (caproate)/mol (ethanol). Besides, since only a small amount of heptanoate and caprylate (<0.1 g/L) were produced, caproate accounted for 98.1-99.9% of total MCCAs produced.

Partially Recovered Chain Elongation after Dilution (Stage III). Dilution is a common strategy to alleviate product inhibition. To test if dilution could resume the activity of chain elongation, half of the reactor volume was replaced with an equal volume of DI water at the beginning of Stage III (Day 18). The replacement of DI water diluted the caproate concentrations in E3 and E5 reactors to 5.74±0.47 g/L (12.68±1.04 g COD/L) and 5.15±0.32 g/L (11.38±0.71 g COD/L). Together with the slight increase of pH (about 0.1), at the beginning of stage III, caproic acid, the undissociated form, in E3 and E5 decreased to 0.36±0.07 and 0.35±0.04 g/L.

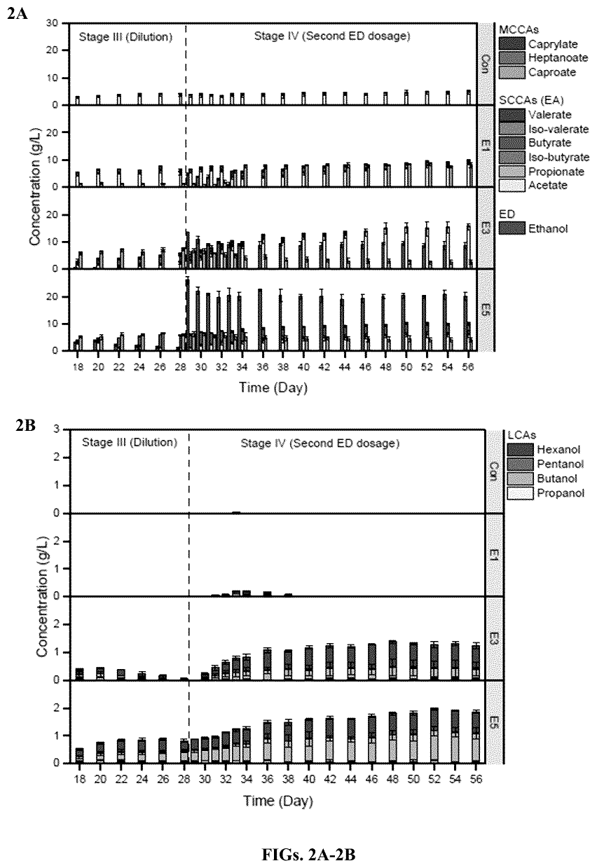

In E3, Dilution Immediately Resumed Both Acetate and Butyrate Production. The concentrations of acetate and butyrate increased from 1.70±0.36 g/L (1.82±0.39 g COD/L) and 1.23±0.54 g/L (2.24±0.98 g COD/L) to 2.37±0.46 g/L (2.54±0.49 g COD/L) and 2.40±0.63 g/L (4.37±1.15 g COD/L) on Day 28, accompanied by ethanol and subsequent long-chain alcohols (LCAs) degradation (FIGS. 2A and 2B). In E5, both acetate and ethanol were gradually consumed to produce butyrate via chain elongation. The concentration of butyrate in E5 increased from 1.01±0.26 g/L (1.82±0.5 g COD/L) to 4.58±0.24 g/L (8.33±0.44 g COD/L) during Stage III. Nevertheless, the chain elongation process in E3 and E5 was largely limited to elongation of acetate into butyrate. Further elongation from butyrate to caproate was rather minor.

Chain Elongation Shifted to LCA Production after Section ED Addition (Stage IV). High ED:EA ratio is another crucial condition to induce MCCA production. Therefore, we hypothesized that the low caproate production in Stage III may be due to lack of ethanol. In Stage IV (chain elongation, Day 29-Day 56), based on the remaining SCCAs (as EA), we dosed ethanol as ED with ED:EA ratios of 1:1, 3:1, and 5:1 into E1, E3, and E5 groups, respectively.

In E3 and E5, the second dosage of ethanol didn't result in further caproate production. Instead, ethanol oxidization was coupled with LCA production (FIG. 2B). Moreover, butyrate and caproate in E3 and E5 gradually decreased during Stage IV while butanol and hexanol were simultaneously formed. Although the consumption of caproate alleviated the toxicity of caproic acid in E3 (0.33±0.07 g/L) and E5 (0.3±0.02 g/L), chain elongation to caproate did not recover. In comparison, since caproate in E1 never reach the inhibition level at the previous stages, the second dose of ethanol successfully induced chain elongation, producing 6.95±0.53 g/L (15.36±1.17 g COD/L) caproate at the end of Stage IV (FIG. 2A).

Microbial Community Structure. Ethanol addition shaped the microbial composition (p≤0.001) (FIG. 3A). Samples collected from various groups of reactors at early stages (I and II) were clustered in the lower left zone of the PCoA plot. After chain elongation (Stages III and IV), samples of the control group formed a separate cluster from E1, E3 and E5 groups, suggesting a distinctive microbial composition after ethanol addition. Further, pairwise comparison revealed significant difference in microbial composition among most groups (p≤0.01), while the difference between E3 and E5 was not significant (p>0.05). Phyla Firmicutes, Chloroflexi, Actinobacteriota, Bacteroidota, and Proteobacteria were dominant in all reactors (FIG. 3B).

So far, 16 strains from 10 genera have been confirmed to be able to produce caproate. Among these microbes, strains in 5 of the 10 genera were found in our samples, i.e., Clostridium, Eubacterium, Ruminococcaceae, Caproiciproducens, and Megasphaera (FIG. 3C). Species belonging to genera Clostridium sensu stricto 1, 7, 10, 12, 13, and 16 were the dominant caproate producers in most of our samples. Some amplicon sequence variants (ASVs) linked to Clostridium sensu stricto 12 matched with Clostridium kluyveri (97.60-98.63% identity) and Clostridium luticellarii (98.63-98.97%), two species known for caproate production. The highest relative abundance of Clostridium (4.0%) in E3 was found on Day 44 (Stage IV) when the LCA production was robust. While in E5, Clostridium was enriched (4.3%) on Day 13 (Stage II) when caproate production reached the highest rate. Dilution (Stage III) and the second dosage of ED (Stage IV) recovered the relative abundance of chain clongators until the LCA production was largely completed. Particularly, Ruminococcaceae and Caproiciproducens strains (97.95% identity with Caproiciproducens galactitolivorans) were barely found in the control group but were widely detected in the E1, E3, and E5 groups during Stages III and IV. We also applied PICRUSt2 to predict MCCA- and LCA-production pathways based on the 16S rRNA gene (Supplementary Information).

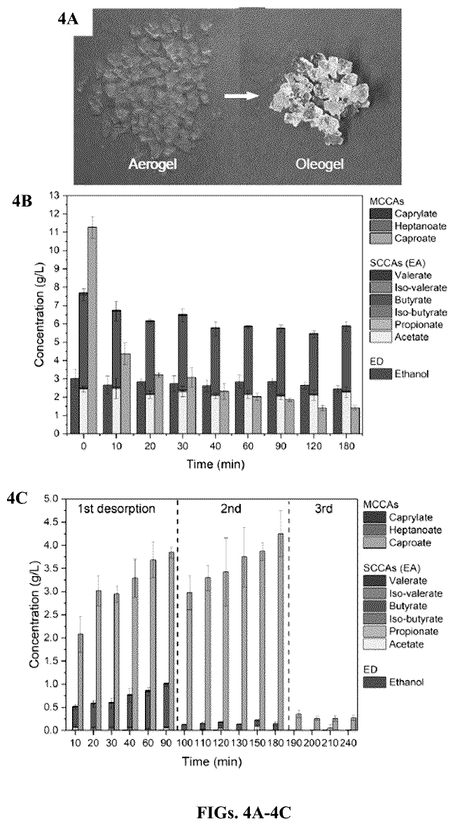

Extraction of MCCAs from Manure Slurry using Oleogel. Olcogel beads (FIG. 4A) exhibited a strong extraction capacity for caproate from real chain elongation broth. Extraction of caproate started quickly (FIG. 4B). The concentration of caproate in the broth decreased from 11.26±0.59 g/L (24.88±1.30 g COD/L) to 1.42±0.13 g/L (3.14±0.29 g COD/L) after 180 min. The absorption efficiency of oleogel for caproate was 87.39±0.96% in 180 min. In comparison, the absorption efficiency for ethanol, acetate, and butyrate were 27.18±7.19%, 6.84±8.16%, and 42.98±11.53%, respectively.

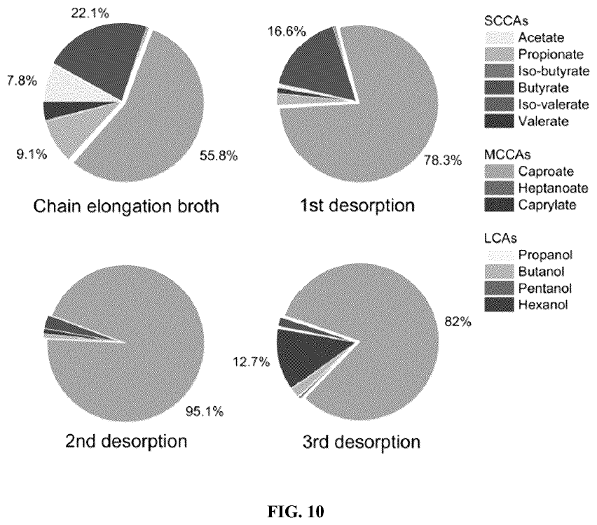

After absorption, the oleogel was washed with DI water for 1 min and then placed in 2-mL pH-12 solutions for desorption (FIG. 4C). Caproate was released from the oleogel beads relatively fast. After 90 min, the concentration of caproate in the solution increased to 3.84±0.12 g/L (8.49±0.27 g COD/L). Another 4.29±0.49 g/L (9.48±1.08 g COD/L) of caproate was released to fresh solution after 90 min during the second round of desorption and 0.27±0.07 g/L (0.60±0.15 g COD/L) of caproate released after 60 min during the third round of desorption. Together, three rounds of desorption recovered 85.26±8.25% of caproate adsorbed. Overall, the extraction of caproate using oleogel increased caproate specificity from 55.78±1.10% in the original chain elongation broth to 78.32±0.41%-95.14±0.36% in the final solutions, with an average cumulative caproate specificity of 86.18±0.32% (FIG. 10).

### Example 3—Supplemental Material

Calculation Methods. The modified Gompertz model was applied to simulate the dynamic parameters of MCCAs production.

$$C = {C}_{max} exp \{ - exp [ \frac{{R}_{max} \times e}{{C}_{max}} ( λ - t ) + 1 ] \} \qquad ( 1 )$$

where C is the cumulative MCCAs concentration (g/L) at time t, Cₘₐₓ is the maximum MCCA concentration (g/L), t is the time (day), Rₘₐₓ is the maximum MCCA production rate (g/L/day), λ is the lag time (day), and e is 2.71828. The simulation was conducted using OriginalPro 2021.

Product yield, selectivity, specificity, and oleogel extraction efficiency were calculated. Product selectivity was calculated as the final carboxylate concentration (mol C) divided by the initial substrate concentration (i.e., initial total concentration of ED and EA in mol C). Product yield was calculated similarly to product selectivity except with different units for the final carboxylate concentration (g/L) and initial substrate concentration (i.e., initial total concentration of ED and EA in g chemical oxygen demand (COD)/L). Product specificity was calculated as the final carboxylate concentrations (mol C) divided by the final total carboxylate and alcohol concentrations (mol C).

$$Product {Yield}^{i} = \frac{{C}_{p , i}}{{\sum }_{j} {C}_{s , j}} \qquad ( 2 )$$

where C_{p,i} is the final concentration of produced species i (g/L), C_{s,j} is the initial concentration of substrate species j (g COD/L).

$$Product {Selectivity}^{i} = \frac{{n}_{i} \times {C}_{p , i}}{{\sum }_{j} ( {n}_{i} \times {C}_{s , j} )} \qquad ( 3 )$$

$$Product {Specificity}^{i} = \frac{{n}_{i} \times {C}_{p , i}}{{\sum }_{j} ( {n}_{j} \times {C}_{p , j} )} \qquad ( 4 )$$

where nᵢ and nⱼ are the carbon numbers of species i and j, C_{p,i} is the final concentration of produced species i (mol/L), C_{s,j} is the initial concentration of substrate species j (mol/L), and C_{p,j} is the final concentration of product species j (mol/L). Extraction efficiency was calculated as the concentration adsorbed by oleogel divided by the initial concentration:

$$Extraction Efficiency = \frac{{C}_{p , i , o} - {C}_{p , i , t}}{{C}_{p , i , o}} \qquad ( 5 )$$

where C_{p,i,0} and C_{p,i,t} are the concentrations of product i in the broth before and after oleogel extraction (g/L).

High-Throughput Sequencing. Manure samples from the 4 groups of 12 reactors were collected at nine time points, totaling 108 samples. Three subsamples of the original manure were also included. Total DNA of these samples was extracted using the DNeasy PowerLyzer PowerSoil Kit (QIAGEN, German) according to the manufacturer's protocols. The V4-V5 hypervariable regions of the 16S rRNA gene were amplified with universal primer pair 515F and 806R and sequenced on the Illumina Miniseq platform at the University of Illinois at Chicago sequencing facility. ASVs were constructed using the Qiime2 workflow. Data2 was applied for sequence quality filtering and ASVs table construction. Taxonomic analysis was performed by comparing it with the SILVA database (silva-138-99-515-806-nb-classifier). PICRUSt2 was used to predict the metabolic pathways based on 16S rRNA marker gene profiles. The functional annotation of PICRUSt2 predictions was obtained based on the Kyoto Encyclopedia of Genes and Genomes (KEGG) database. The full gene name and description of each KO shown in this study can be found in Table 3. The results were presented as means and standard deviations from triplicate biological samples.

TABLE 3

| | |
|---|---|
| KO number | Gene and description [Enzyme number] |

Reverse beta oxidation pathways

| | |
|---|---|
| K00632 | fadA, fadI; acetyl-CoA acyltransferase [EC: 2.3.1.16] |
| K07508 | ACAA2; acetyl-CoA acyltransferase 2 [EC: 2.3.1.16] |
| K07509 | HADHB; acetyl-CoA acyltransferase [EC: 2.3.1.16] |
| K07516 | fadN; 3-hydroxyacyl-CoA dehydrogenase [EC: 1.1.1.35] |
| K01825 | fadB; 3-hydroxyacyl-CoA dehydrogenase/enoyl-CoA hydratase/3-hydroxybutyryl-CoA |
| | epimerase/enoyl-CoA isomerase [EC: 1.1.1.35]/[EC: 4.2.1.17]/[EC: 5.1.2.3]/[EC: 5.3.3.8] |
| K01782 | fadJ; 3-hydroxyacyl-CoA dehydrogenase/enoyl-CoA hydratase/3-hydroxybutyryl-CoA |
| | epimerase [EC: 1.1.1.35]/[EC: 4.2.1.17]/[EC: 5.1.2.3] |
| K01692 | paaF, echA; enoyl-CoA hydratase [EC: 4.2.1.17] |
| K13767 | fadB; enoyl-CoA hydratase [EC: 4.2.1.17] |
| K00248 | ACADS, bed; butyryl-CoA dehydrogenase [EC: 1.3.8.1] |
| K06445 | fadE; acyl-CoA dehydrogenase [EC: 1.3.99.—] |
| K09478 | ACADSB: short/branched chain acyl-CoA dehydrogenase [EC: 1.3.99.12] |
| K00626 | E2.3.1.9, atoB; acetyl-CoA C-acetyltransferase [EC: 2.3.1.9] |
| K10806 | yciA; acyl-CoA thioesterase YciA [EC: 3.1.2.—] |
| K02614 | paaI; acyl-CoA thioesterase [EC: 3.1.2.—] |
| K10804 | tesA; acyl-CoA thioesterase I [EC: 3.1.2.—]/[EC: 3.1.1.5] |
| K10805 | tesB; acyl-CoA thioesterase II [EC: 3.1.2.—] |

Acetyl-CoA or LCA production

| | |
|---|---|
| K00001 | E1.1.1.1, adh; alcohol dehydrogenase [EC: 1.1.1.1] |
| K00002 | AKR1A1, adh; alcohol dehydrogenase (NADP+) [EC: 1.1.1.2] |
| K00128 | ALDH; aldehyde dehydrogenase (NAD+) [EC: 1.2.1.3] |

Fatty acid biosynthesis

| | |
|---|---|
| K01962 | accA; acetyl-CoA carboxylase carboxyl transferase subunit alpha [EC: 6.4.1.2]/[EC: 2.1.3.15] |
| K02160 | accB, bccP; acetyl-CoA carboxylase biotin carboxyl carrier protein |
| K01961 | accC; acetyl-CoA carboxylase, biotin carboxylase subunit [EC: 6.4.1.2]/[EC: 6.3.4.14] |
| K01963 | accD; acetyl-CoA carboxylase carboxyl transferase subunit beta [EC: 6.4.1.2] [EC: 2.1.3.15] |
| K11263 | bccA, pccA; acetyl-CoA/propionyl-CoA carboxylase, biotin carboxylate, biotin carboxyl |
| | carrier protein [EC: 6.4.1.2]/[EC: 6.4.1.3]/[EC: 6.3.4.14] |
| K18473 | fabY; acetoacetyl-[acyl-carrier protein] synthase [EC: 2.3.1.180] |
| K11533 | fas; fatty acid synthase, bacteria type [EC: 2.3.1.—] |
| K00645 | fabD; [acyl-carrier-protein] S-malonyltransferase [EC: 2.3.1.39] |
| K00647 | fabB; 3-oxoacyl-[acyl-carrier-protein] synthase I [EC: 2.3.1.41] |
| K09458 | fabF; 3-oxoacyl-[acyl-carrier-protein] synthase II [EC: 2.3.1.179] |
| K00059 | fabG; 3-oxoacyl-[acyl-carrier protein] reductase [EC: 1.1.1.100] |
| K01716 | fabA; 3-hydroxyacyl-[acyl-carrier protein] dehydratase/trans-2-decenoyl-[acyl-carrier |
| | protein] isomerase [EC: 4.2.1.59 5.3.3.14] |
| K02372 | fabZ; 3-hydroxyacyl-[acyl-carrier-protein] dehydratase [EC: 4.2.1.59] |
| K16363 | lpxC-fabZ; UDP-3-O-[3-hydroxymyristoyl] N-acetylglucosamine deacetylase/3- |
| | hydroxyacyl-[acyl-carrier-protein] dehydratase [EC: 3.5.1.108 4.2.1.59] |
| K00208 | fabI; enoyl-[acyl-carrier protein] reductase I [EC: 1.3.1.9 1.3.1.10] |
| K02371 | fabK; enoyl-[acyl-carrier protein] reductase II [EC: 1.3.1.9] |

Detailed Analytical Procedures. For alcohols, SCCAs, and MCCAs measurement, the carrier gas was nitrogen at a flow rate of 30 mL/min. The inlet and detector temperatures were set at 260° C. The column operating temperature profiles were 80° C. for 1 min, 120° C. for 2 min (increasing at 20° C./min), and 205° C. for 5 min (increasing at 10° C./min). The liquid samples were acidified with 3% (v/v) formic acid before analysis. The gas composition (CO₂, hydrogen (H₂), and methane (CH₄)) was measured by a micro gas chromatograph (Micro GC 490, Agilent, California, USA) equipped with a thermal conductivity detector. The temperatures of the injector and column were kept at 60 and 75° C., respectively. Helium was used as the carrier gas at a flow rate of 25 mL/min. Protein was measured using the BCA Protein Assay Kit (Thermo Fisher Scientific, Massachusetts, USA). Soluble carbohydrate was measured using the sulfuric acid-phenol method. The TS and VS were determined according to the Standard Methods.

Supplementary Details of Alcohol and Acid Production. At Stage II, Long-chain alcohols (LCAs), such as butanol and hexanol, were also produced and accumulated in E3 and E5 in small amounts (FIG. 7). The concentrations of butanol and hexanol were 0.37±0.19 g/L (0.96±0.49 g COD/L) and 0.34±0.00 g/L (0.96±0.00 g COD/L) in E3, and 0.35±0.11 g/L (0.91±0.29 g COD/L) and 0.38±0.09 g/L (1.07±0.25 g COD/L) in E5 on Day 12, respectively.

At the end of Stage III, the concentration of caproate in E3 and E5 only increased 1.54±0.03 g/L (3.40±0.07 g COD/L) and 0.80±0.49 g/L (1.77±1.08 g COD/L). Due to the regeneration of SCCAs at the end of stage III, the pH of the E3 and E5 groups dropped to 5.6±0.1 and 5.5±0.0, resulting in more than 1 g/L of undissociated caproate in both groups.

At Stage IV, ethanol was oxidized to 15.40±1.05 g/L (16.48±1.12 g COD/L) acetate in E3 and 6.18±1.22 g/L (6.61±1.31 g COD/L) acetate in E5. The concentrations of butanol and hexanol were 0.33±0.23 g/L (0.86±0.60 g COD/L) and 0.80±0.11 g/L (2.26±0.31 g COD/L) in the E3 group and 1.00±0.21 g/L (2.60±0.55 g COD/L) and 0.76±0.06 g/L (2.14±0.17 g COD/L) in the E5 group.

Microbial Community Structure at the Phylum Level. At the phylum level (FIG. 4B), Firmicutes, Chloroflexi, Actinobacteriota, Bacteroidota, and Proteobacteria were dominant in all reactors. The overall abundances of these phyla were relatively stable. While in the E1, E3, and E5 groups, Firmicutes, the only known phylum containing chain clongators, as well as Actinobacteriota and Proteobacteria, were enriched over time. In contrast, the relative abundances of Chloroflexi and Bacteroidota gradually decreased during the chain elongation process.

Predicted Chain Elongation Functions. We applied PICRUSt2 to predict MCCA- and LCA-production pathways based on the 16S rRNA gene. A total of 16 genes involved in the reverse β-oxidation (RBO) pathway were predicted (FIG. 8A). atoB (K00626) was the dominant gene of the RBO pathway, followed by paaF, echA (K01692), and fadA, fadI (K00632). atoB (K00626) and fadA, fadI (K00632) encode thiolase, which involves in the first step of the RBO pathway. paaF (K01692) encodes enoyl-CoA reductase, an enzyme converting (Cₙ₊₂)-Enoyl-CoA into (Cₙ₊₂)-Acyl-CoA during the RBO cycle. ED:EA ratios of 3:1 and 5:1 induced a higher relative abundance of genes involved in the RBO pathway at stage IV (p<0.05) (FIG. 8B).

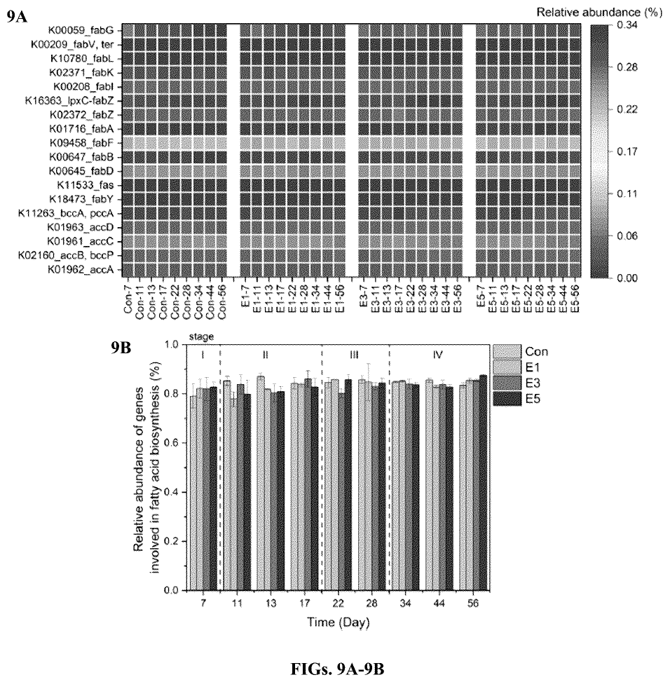

Besides, 18 other genes involved in the fatty acid biosynthesis pathway (FAB) were also predicted (FIG. 9). However, the total relative abundance of FAB genes was stable during the whole process and had no differences among the 4 groups. Therefore, caproate production was more likely linked to the RBO rather than the FAB pathway. Three other genes responsible for acetyl CoA and/or alcohol production showed higher relative abundance in E1, E3 and E5 at Stage IV, coincident with robust LCA production (FIG. 8C).

Environmental Implications. In summary, we investigated the feasibility of converting the organics in cattle manure slurry to caproate. Compared with other organic wastes, cattle manure exhibited excellent performance in terms of titer and yield. This is due to the intrinsic diversity of chain elongators in cattle manure, which is probably derived from the rumen microbiome. We also developed an oleogel extraction technology to effectively recover caproate from real cattle manure chain elongation broths. The complete treatment train that includes both the production and recovery of caproate make the technologies developed in this study feasible for adoption by stakeholders to valorise a significant agricultural waste into a valuable bioproduct.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of making medium chain carboxylic acids (MCCAs; C6-C12), comprising:
providing a feedstock, wherein the feedstock is cattle manure or wastewater from cattle manure holding ponds;
combining the feedstock with material providing an electron donor at a suitable ratio;
fermenting the combination of the feedstock with the material providing an electron donor under first appropriate conditions in fermentation broth to produce short chain fatty acids (SCFAs);
elongating the SCFAs under second appropriate conditions into medium chain carboxylic acids (MCCAs); and
extracting the MCCAs.

2. The method of claim 1, wherein the material providing the electron donor is corn silage.

3. The method of claim 1, wherein the material providing the electron donor is ethanol, lactate, or combinations thereof.

4. The method of claim 1, wherein the suitable ratio of the feedstock with the material providing an electron donor is from about 1:3 to about 1:5.

5. The method of claim 1, wherein the MCCAs comprise caproate.

6. The method of claim 1, wherein the extraction of MCCAs is selective for caproate.

7. The method of claim 1, wherein the extraction of caproate uses oleogel beads.

8. The method of claim 1, further comprising adding additional fermentation broth during or following the elongating step.

9. The method of claim 8, further comprising adding additional material providing an electron donor to the additional fermentation broth.

10. The method of claim 1, wherein the method does not require exogenous enzymes for chain elongation.

11. The method of claim 1, wherein the method relies on bacteria intrinsic to the feedstock for chain elongation.

12. The method of claim 11, wherein the bacteria intrinsic to the feedstock are Clostridium, Eubacterium, Ruminococcaceae, or Caproiciproducens.

13. The method of claim 1, wherein the method does not require pH adjustment.

14. A system for making medium chain carboxylic acids (MCCAs) comprising:
a first reactor, wherein the input is cattle manure or wastewater from cattle manure holding ponds and the output is short chain fatty acids (SCFAs); and
a second reactor, wherein the input is the SCFAs produced in the first reactor and the output is MCFAs.

15. The system of claim 14, wherein the output from the second reactor is caproate.

16. Oleogel beads comprising aerogel and at least one organic solvent.

17. The oleogel beads of claim 16, wherein the organic solvent is immiscible with water and has low polarity.

18. The oleogel beads of claim 16, wherein the organic solvent is dodecane.

19. The oleogel beads of claim 16, wherein the organic solvent further comprises trioctylphosphine oxide (TOPO) at about 1.5% to about 6%.

20. The oleogel beads of claim 16, wherein the aerogel has a particle size of about 1.2 mm to about 4.0 mm.

21. The oleogel beads of claim 16, wherein the aerogel has a pore diameter of about 15 nm to about 25 nm.

22. A method of obtaining high yields of caproate from a fermentation broth comprising MCCAs, the method comprising:
contacting the fermentation broth comprising MCCAs with the oleogel beads of claim 16, thereby obtaining high recovery yields of caproate from the fermentation broth.
